(19) United States
(12) Patent Application Publication
(10) Pub. No.: US 2024/0192184 A1
(43) Pub. Date: Jun. 13, 2024

(54) DETECTION OF FLAVOUR LOSS

(71) Applicant: Givaudan SA, VERNIER (CH)

(72) Inventor: Stephen A. FERGUSON, Cincinnati, OH (US)

(21) Appl. No.: 18/553,558

(22) PCT Filed: Apr. 8, 2022
(86) PCT No.: PCT/EP2022/059448

§ 371 Date: Sep. 30, 2023

Related U.S. Application Data
(60) Provisional application No. 63/174,477, filed on Apr. 13, 2021.

(51) Int. Cl. G01N 30/88; G01N 1/22; G01N 1/28; G01N 1/38; G01N 1/40; G01N 1/44; G01N 30/02

(52) U.S. Cl. CPC G01N 30/88; G01N 1/44; G01N 2001/2229; G01N 2001/2866; G01N 2001/2873; G01N 2001/386; G01N 1/405; G01N 2030/025

(57) ABSTRACT

Disclosed is method for detecting the amount of flavour loss from a flavour compound of flavour composition containing one or more flavour compounds over time. The method utilizes a combination of dynamic headspace sampling, chromatography and mass spectrometry. The method of detecting the flavour loss over time enables an accurate prediction of the shelf-life a particular flavour compound or flavour composition.

### TECHNICAL FIELD

Disclosed is a method for detecting flavour loss from a flavour compound or composition containing at least one flavour compound. More particularly, disclosed is a method for detecting flavour loss from an encapsulated flavour compound or flavour composition containing at least one volatile flavour compound to provide an approximation of overall stability and shelf-life of the flavour compound or composition.

### BACKGROUND

Flavours play an important role in overall consumer satisfaction in the consumption of foods and beverages. To minimize or prevent degradation or loss of flavour characteristics during processing and storage, volatile flavour compounds and compositions may be encapsulated prior to incorporation into a food or beverage product.

Encapsulation is a process by which a volatile flavour compound or a flavour composition containing a volatile flavour compound and/or other materials is coated, covered, or encapsulated within another material. The encapsulated flavour material is generally referred to as the core material and the outer encapsulating material is referred to as the shell, wall material, or encapsulant. Encapsulation of volatile flavour compounds or compositions may be achieved by different methods including spray drying, spray chilling, spray cooling, freeze drying, coacervation, extrusion, and molecular inclusion.

The result of the encapsulation process is the formation of microcapsules containing a flavour compound or composition as the core material surrounded by an outer shell or wall that isolates or otherwise protects the core flavour material from the external environment. Encapsulation protects the encapsulated flavour compound from degradation by preventing oxidation reactions and/or volatile loss over time. Oxidation is a well-known degradation pathway for flavour compounds, oils and other food ingredients and is routinely examined in connection with shelf-life determinations. Volatile loss is another potential degradation pathway for certain flavour compounds, but is not typically examined in connection with shelf-life determinations.

There remains a need in the art to provide a method by which to determine loss of flavour by oxidative pathways and volatilization of flavour compounds, and to thereby more accurately enable shelf-life determination for flavour compounds.

### SUMMARY

Disclosed is a method for detecting flavour loss from a flavour compound comprising introducing a liquid sample containing a flavor compound into a sealed sample vessel, heating the liquid sample containing the flavour compound to produce a gaseous analyte, flowing an inert carrier gas through the sealed sample vessel, collecting the gaseous analyte, separating flavour components derived from the flavour compound contained in the gaseous analyte, identifying the separated flavour components of the analyte and quantifying each of the separated flavour components.

### BRIEF DESCRIPTION OF THE DRAWINGS

FIG. 1 is a graph showing the calibration curve used to monitor headspace limonene concentration over time when stored under accelerated storage conditions and with constant air flow.

FIG. 2 is a graph showing the limonene concentration when stored at 60° C. over a period of days with constant air flow.

FIG. 3 is a graph showing the concentration of cymene, a degradation product of limonene, when limonene is stored at 60° C. over a period of days with constant air flow. The formation of cymene is indicative of degradation of limonene and flavour loss.

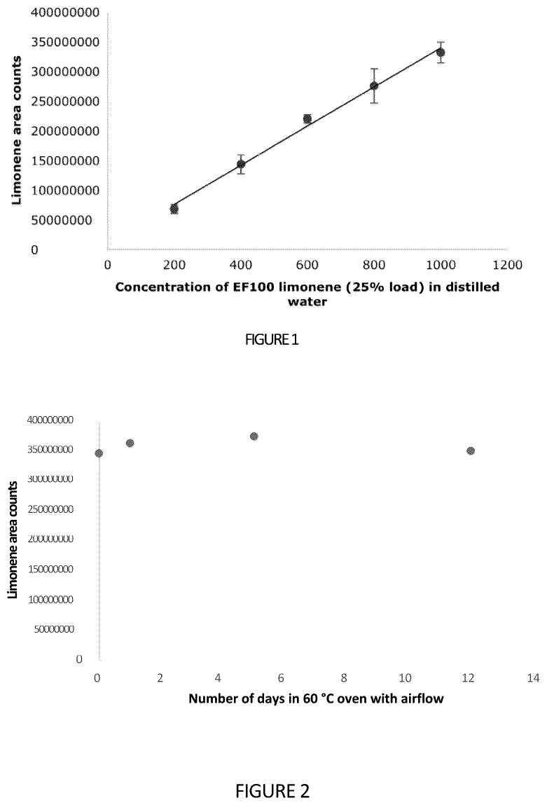

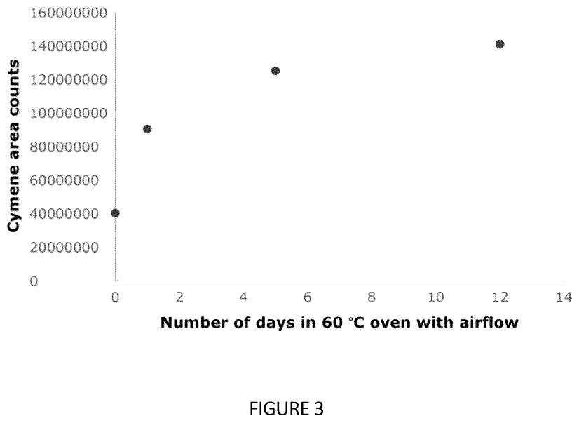

### DETAILED DESCRIPTION

The following text sets forth a broad description of numerous illustrative embodiments of the present disclosure. The description is to be construed as exemplary only and does not describe every possible embodiment since describing every possible embodiment would be impractical, if not impossible. It will be understood that any feature, characteristic, component, composition, ingredient, product, step or methodology described herein can be deleted, combined with or substituted for, in whole or part, any other feature, characteristic, component, composition, ingredient, product, step or methodology described herein. Numerous alternative embodiments could be implemented, using either current technology or technology developed after the filing date of this patent, which would still fall within the scope of the claims. All publications and patents cited herein are incorporated herein by reference.

Disclosed is a method for detecting the loss of flavour from a sample of a flavour compound, such as a sample of an encapsulated flavour compound, to assess the extent of degradation of the flavour that can be attributed to volatilization and/or oxidation of the flavour compound. The method involves the combined use of dynamic headspace sampling, gas chromatography and mass spectrometry to quantify volatile flavor components as well as their degradation products.

The method for detecting flavour loss from a flavour compound comprises providing a flavour compound or flavour composition comprising one or more flavour compounds to be analyzed for degradation. The flavour compound or flavour composition comprising one or more flavour compounds may be analyzed for degradation from chemical oxidation and/or volatilization.

The encapsulated flavour compound or flavour composition comprising one or more flavour compounds is provided for the analysis. The flavour compound or composition is contained in an immiscible fluid to provide a liquid sample comprising the one or more flavour compounds for the analysis. According to certain embodiments, the suitable fluid comprises a solvent. According to certain embodiments, the solvent comprises water. The flavour compound may be provided in a dried powder form having a water-miscible outer shell and a water-immiscible inner core. The water-immiscible inner core comprises an oil containing the flavour compound. A desired quantity of the dried powder flavour compound is combined with water to dissolve the outer water-miscible shell, thereby leaving behind oil droplets containing the flavour compound dispersed within the water. The liquid sample comprising the flavour compound(s) is introduced into a sealed sample vessel, such as a sealed vial. The sealed sample vessel comprises an interior having a liquid sample region located at the bottom of the vessel and a headspace region located above the liquid sample region. According to certain embodiments, the flavour compound or flavour composition comprising one or more flavor compounds may be placed within the interior of the sample vessel. According to this embodiment, the solvent is also introduced into sample vessel and the flavour components are dissolved in the solvent in the sample vessel.

According to certain embodiments, the encapsulated flavour compound or composition may need to be further processed to release the flavour components or compounds from the microcapsule. Such further processing includes mechanically breaking apart, comminuting, cracking, crushing, cutting, dividing, fracturing, grating, grinding, or splitting the microcapsules to release the flavour components.

The method further comprises heating the liquid sample containing the dissolved flavour compound(s) at a temperature and for a time sufficient to produce a gaseous analyte. After or during the sample heating process, an inert carrier gas is flowed through the sealed sample vessel to carry the gaseous analyte from the headspace region of the interior of the sealed sample vessel to a collection vessel.

The step of flowing the inert carrier gas through the sealed sample vessel comprises flowing the inert gas into from the environment external to the sealed sample vessel into the headspace of the sealed sample vessel through an inlet. According to certain embodiments, the inlet comprises an elongated structure configured to receive an inert carrier gas from a source of carrier gas and to permit passage of the inert carrier gas through the inlet to a desired location, such as the interior of a sealed sample vessel. According to certain embodiments, the elongated inlet structure may be selected from the group consisting of a cannula, conduit, cylinder, duct, hose, line, needle, passage, or tube. According to certain embodiments, the elongated inlet comprises a needle with a tip configured for penetrating or otherwise piercing a surface of the sealed sample vessel. The inert carrier gas is continuously flowed into the headspace region of the sealed sample vessel and the flow of the carrier gas continuously collects flavour components within the headspace region for a desired period of time.

The step of flowing the inert carrier gas through the sealed sample vessel further comprises flowing the inert gas containing the analyte from the headspace region of the sealed sample vessel to the external environment of the sealed sample vessel. According to certain embodiments, the outlet comprises an elongated structure configured to receive inert carrier gas and gaseous analyte from the headspace region of the sealed sample vessel and to permit passage of the combination of the inert carrier gas and gaseous analyte through the outlet to a desired location external to the sealed sample vessel. According to certain embodiments, the elongated outlet structure may be selected from the group consisting of a cannula, conduit, cylinder, duct, hose, line, needle, passage, or tube. According to certain embodiments, the elongated outlet comprises a needle with a tip configured for penetrating or otherwise piercing a surface of the sealed sample vessel.

The method further comprises the step of collecting the flavour components contained in the gaseous analyte for subsequent separation, identification and quantification. The step of collecting the flavour components of the gaseous analyte comprises flowing the inert carrier gas containing gaseous analyte from the headspace region of the sealed sample vessel to a collection vessel through the outlet that is in fluid communication with the collection vessel. The collection vessel includes an adsorbent for adsorbing the flavour components of the gaseous analyte. The headspace components are thus directed toward the collection vessel at least partially filled with an adsorbent material where the headspace components are collected and subsequently desorbed for transfer into a gas chromatography column.

Any adsorbent that is capable of collecting by adsorption the desired flavour components contained within the analyte and from which the flavour components can be subsequently desorbed may be used as the adsorbent material in the disclosed method. According to certain embodiments, the method step of collecting the flavour components from the analyte comprises adsorbing the flavour components on an adsorbent polymeric material positioned within the collection vessel. Without limitation, and only by way of illustration, the adsorbent material may be selected from carbon molecular sieves, spherical graphitized polymer carbon, graphitized carbon black and porous polymer resins. According to certain embodiments, the adsorbent material comprises a carbon molecular sieve. A suitable carbon molecular sieve material is commercially available under the registered trademark CARBOXEN® (Sigma-Aldrich Co. LLC, St. Louis, Missouri, US). According to other illustrative embodiments, the adsorbent material comprises a porous polymer resin. A suitable porous polymer resin is commercially available under the registered trademark TENAX® (Sigma-Aldrich Co. LLC, St. Louis, Missouri, US), and is based on 2,6-diphenyl-p-phenylene oxide.

The flavour components from the analyte are desorbed from the adsorbent polymeric material for separation, identification and quantification. According to certain embodiments, the step of desorbing the flavour components from the adsorbent polymeric material comprises heating the collection vessel to a temperature and for a time sufficient to at least partially vaporize flavour components of the analyte which results in desorption of the flavour components from the polymeric adsorbent.

The flavour components from the analyte that have been desorbed from the polymeric adsorbent are carried by an inert carrier gas into a gas chromatograph for separation by gas chromatography.

The method further comprises identifying and quantifying the separated flavour components by mass spectrometry. The amount of degradation product derived from the flavour compound is quantified. According to certain embodiments, the amount of degradation product formed by oxidation of the flavour compound is quantified. According to certain embodiments, the amount of degradation product formed by volatization of the flavour compound is quantified.

Examples of suitable flavour ingredients include, but are not limited to, natural flavours, artificial flavours, synthetic flavour oils and flavoring aromatics and/or oils, oleoresins, essences, and distillates, and a combination comprising at least one of the foregoing.

Flavour oils include spearmint oil, cinnamon oil, oil of wintergreen (methyl salicylate), peppermint oil, Japanese mint oil, clove oil, bay oil, anise oil, eucalyptus oil, thyme oil, cedar leaf oil, oil of nutmeg, allspice, oil of sage, mace, oil of bitter almonds, and cassia oil; useful flavoring agents include artificial, natural and synthetic fruit flavours such as vanilla, and citrus oils including lemon, orange, lime, grapefruit, yuzu, sudachi, myrcenol, sinensal, bergamot oil, and fruit essences including apple, pear, peach, grape, raspberry, blackberry, gooseberry, blueberry, strawberry, cherry, plum, prune, raisin, cola, guarana, neroli, pineapple, apricot, banana, melon, apricot, cherry, tropical fruit, mango, mangosteen, pomegranate, papaya, and so forth.

Additional exemplary flavours imparted by a flavour ingredient include a milk flavour, a butter flavour, a cheese flavour, a cream flavour, and a yogurt flavour, a vanilla flavour, teaflavours, such as a green tea flavour, an oolong tea flavour, a cocoa flavour, a chocolate flavour, and a coffee flavour; mint flavours, such as a peppermint flavour, a spearmint flavour, and a Japanese mint flavour; spicy flavours, such as an asafetida flavour, an ajowan flavour, an anise flavour, an angelica flavour, a fennel flavour, an allspice flavour, a cinnamon flavour, a chamomile flavour, a mustard flavour, a cardamom flavour, a caraway flavour, a cumin flavour, a clove flavour, a pepper flavour, a coriander flavour, a sassafras flavour, a savory flavour, a Zanthoxyli Fructus flavour, a perilla flavour, a juniper berry flavour, a ginger flavour, a star anise flavour, a horseradish flavour, a thyme flavour, a tarragon flavour, a dill flavour, a capsicum flavour, a nutmeg flavour, a basil flavour, a parsley flavour, a marjoram flavour, a rosemary flavour, a bayleaf flavour, and a wasabi (Japanese horseradish) flavour; a nut flavour such as an almond flavour, a hazelnut flavour, a macadamia nut flavour, a peanut flavour, a pecan flavour, a pistachio flavour, and a walnut flavour; floral flavours; and vegetable flavours, such as an onion flavour, a garlic flavour, a cabbage flavour, a carrot flavour, a celery flavour, mushroom flavour, and a tomato flavour.

According to some embodiments, flavour ingredients may also include aldehydes and esters such as cinnamyl acetate ((E)-3-phenylprop-2-en-1-yl acetate); cinnamaldehyde ((2E)-3-phenylprop-2-enal); citral diethylacetal ((E)-1,1-dimethoxy-3,7-dimethylocta-2,6-diene), dihydrocarvyl acetate (2-methyl-5-prop-1-en-2-ylcyclohexyl acetate), eugenyl formate ((2S)-1,3,3-trimethylbicyclo[2.2.1]heptan-2-yl acetate), p-methylanisol (1-methoxy-4-methylbenzene), and so forth can be used. Further examples of aldehyde flavourings include acetaldehyde (apple), benzaldehyde (cherry, almond), anisic aldehyde (4-methoxybenzaldehyde) (licorice, anise), cinnamic aldehyde ((2E)-3-phenylprop-2-enal) (cinnamon), citral (E)-3,7-dimethylocta-2,6-dienal), i.e., alpha-citral ((EE)-3,7-dimethylocta-2,6-dienal (lemon, lime), neral, i.e., beta-citral ((EZ)-3,7-dimethylocta-2,6-dienal (lemon, lime), decanal (orange, lemon), ethyl vanillin (vanilla, cream), heliotrope, i.e., piperonal (benzo[d][1,3]dioxole-5-carbaldehyde) (vanilla, cream), vanillin (vanilla, cream), alpha-amyl cinnamaldehyde ((E or Z)-2-benzylideneheptanal) (spicy fruity flavours), butyraldehyde (butanal) (butter, cheese), valeraldehyde (pentanal) (butter, cheese), citronellal (3,7-dimethyloct-6-enal) (modifies, many types), decanal (citrus fruits), aldehyde C-8 (octanal) (citrus fruits), aldehyde C-9 (nonanal) (citrus fruits), aldehyde C-12 (dodecanal) (citrus fruits), 2-ethyl butyraldehyde (2-ethylbutanal) (berry fruits), hexenal, i.e., trans-2 hexenal (berry fruits), tolyl aldehyde (4-methylbenzaldehyde) (cherry, almond), veratraldehyde (3,4-dimethoxybenzaldehyde) (vanilla), 2,6-dimethyl-5-heptenal, i.e., melonal (melon), 2,6-dimethyloctanal (green fruit), and 2-dodecenal (citrus, mandarin), and the like.

In another embodiment, flavour ingredients include natural compounds derived from Maillard reactions. Examples of suitable fragrance ingredients include, but are not limited to hex-3-en-1-yl butyrate; 2-methyl-1-phenylpropan-2-yl acetate; 2-methyl-1-phenylpropan-2-yl butyrate; 4-(tert-butyl)cyclohexyl acetate; undecan-2-one; 2-benzylideneoctanal; 3,7-dimethylnona-1,6-dien-3-yl acetate; 3,7-dimethylocta-2,6-dien-1-yl acetate; 3,7-dimethylocta-2,6-dienal; non-6-enal; tridec-2-enenitrile; 1-((1,8a)-1,4,4,6-tetramethyl-2,3,3a,4,5,8-hexahydro-1-5,8a-methanoazulen-7-yl)ethanone; 1-(2,3,8,8-tetramethyl-1,2,3,4,5,6,7,8-octahydronaphthalen-2-yl)ethanone; 1-butoxy-1-oxopropan-2-yl butyrate; 2-methyl-1-phenylpropan-2-ol; allyl 2-(isopentyloxy)acetate; allyl 3-cyclohexylpropanoate; methyl non-2-ynoate; undec-9-enal; 1,3,4,5,6,7-hexahydro-. beta., 1,1,5,5-pentamethyl-2-2,4a-ethanonaphthalene-8-ethanol; 1-(1-ethoxyethoxy)hex-3-ene; 1-(2,6,6-trimethylcyclohex-3-en-1-yl)but-2-en-1-one; 1-(2,6,6-trimethylcyclohexa-1,3-dien-1-yl)but-2-en-1-one; 1,1-diethoxy-3,7-dimethylocta-2,6-diene; 2-ethyl-4-(2,2,3-trimethylcyclopent-3-en-1-yl)but-2-en-1-ol; 3,7-dimethylnona-1,6-dien-3-ol; 3,7-dimethylocta-2,6-dien-1-yl isobutyrate; 3-methyl-2-(pent-2-en-1-yl)cyclopent-2-enone; 4-(2,5,6,6-tetramethylcyclohex-2-en-1-yl)but-3-en-2-one; 4-(2,6,6-trimethylcyclohex-1-en-1-yl)but-3-en-2-one; 4-(2,6,6-trimethylcyclohex-2-en-1-yl)but-3-en-2-one; 1,3,3-trimethylbicyclo[2.2.1]heptan-2-yl acetate; (2,4)-1,7,7-trimethylbicyclo[2.2.1]heptan-2-yl acetate; (ethoxymethoxy)cyclododecane; 1-(2,6,6-trimethylcyclohexa-1,3-dien-1-yl)but-2-en-1-one; 3,4,5,6,6-pentamethylhept-3-en-2-one; 3,7,11-trimethyldodeca-1,6,10-trien-3-yl acetate; 3,7-dimethylocta-2,6-dien-1-ol; 3,7-dimethylocta-2,6-dienal; 3-methyl-5-(2,2,3-trimethylcyclopent-3-en-1-yl)pent-4-en-2-ol; 3-methylcyclotetradec-5-enone; 4-((3a, 7a)-hexahydro-1-4,7-methanoinden-5(6)-ylidene)butanal; 4-(2,6,6-trimethylcyclohex-2-en-1-yl)but-3-en-2-one; 4,11,11-trimethyl-8-methylenebicyclo[7.2.0]undec-4-ene; 4-methyldec-3-en-5-ol; 5-methylheptan-3-one oxime; methyl non-2-enoate; oxacyclohexadec-12-en-2-one; 1-((2-(tert-butyl)cyclohexyl)oxy)butan-2-ol; 1-(3,3-dimethylcyclohex-1-en-1-yl)pent-4-en-1-one; 1,2,4)-1,3,3-trimethylbicyclo[2.2.1]heptan-2-ol; 1,2,4)-2′-isopropyl-1,7,7-trimethylspiro[bicyclo[2.2.1]heptane-2,4′-[1,3]dioxane]; 1,2,5)-2-ethoxy-2,6,6-trimethyl-9-methylenebicyclo[3.3.1]nonane; 112) (4-(4-hydroxyphenyl)butan-2-one; 1-methyl-2-(5-methylhex-4-en-2-yl)cyclopropyl)-methanol; 1-methyl-4-(4-methylpent-3-en-1-yl)cyclohex-3-enecarbaldehyde; 1-methyl-4-propan-2-ylcyclohexa-1,4-diene; (1s,4s)-1,3,3-trimethyl-2-oxabicyclo[2.2.2]octane; 2-(4-methylcyclohex-3-en-1-yl)propan-2-yl acetate; 2)-ethyl 3-isopropylbicyclo[2.2.1]hept-5-ene-2-carboxylate; 2,2-dimethoxyethyl)benzene; 2,6)-3,7-dimethylnona-2,6-dienenitrile; 2,6-dimethyloctan-2-ol; 2-isopropyl-5-methylcyclohexanol; 2-methyl-4-oxo-4-pyran-3-yl isobutyrate; 2-methyl-6-methyleneoct-7-en-2-yl acetate; 3-(4-isopropylphenyl)-2-methylpropanal; 3,5,5-trimethylhexyl acetate; 3,5-dimethylhex-3-en-2-yl)oxy)-2-methylpropyl cyclopropanecarboxylate; 3,7-dimethyloct-6-en-1-yl acetate; 3,7-dimethyloct-6-en-1-yl formate; 3,7-dimethyloct-6-en-1-yl propionate; 3,7-dimethyloct-6-enenitrile; 3,7-dimethylocta-1,6-dien-3-yl acetate; (3a,4,7,7a)-ethyl octahydro-1-4,7-methanoindene-3a-carboxylate; (3a,6,7a)-3a,4,5,6,7,7a-hexahydro-1-4,7-methanoinden-6-yl acetate; (3a,6,7a)-3a,4,5,6,7,7a-hexahydro-1-4,7-methanoinden-6-yl isobutyrate; (3a,6,7a)-3a,4,5,6,7,7a-hexahydro-1-4,7-methanoinden-6-yl propionate; (3-methyl-2-pentylcyclopent-2-enone; 4,7-dimethyloct-6-en-3-one; 4-methylene-2-phenyltetrahydro-2-pyran; 6,6-dimethoxy-2,5,5-trimethylhex-2-ene; allyl heptanoate; cyclohexyl 2-hydroxybenzoate; ethyl 2,6,6-trimethylcyclohexa-1,3-diene-1-carboxylate; ethyl heptanoate; ethyl hexanoate; hexyl isobutyrate; pentyl 2-hydroxybenzoate; propanedioic acid 1-(1-(3,3-dimethylcyclohexyl)ethyl) 3-ethyl ester; 3-methyl-4-(2,6,6-trimethylcyclohex-2-en-1-yl)but-3-en-2-one; 1-(3,3-dimethylcyclohexyl)ethyl formate; 1-(3,5,5,6,8,8-hexamethyl-5,6,7,8-tetrahydronaphthalen-2-yl)ethanone; 1-(spiro[4.5]dec-6-en-7-yl)pent-4-en-1-one; 1,1,2,3,3-pentamethyl-2,3,6,7-tetrahydro-1-inden-4(5)-one; decanal; 2-methyldecanal; undec-10-enal; undecanal; 2-methylundecanal; 1-methyl-4-(prop-1-en-2-yl)cyclohex-1-ene; 1-methyl-4-(prop-1-en-2-yl)cyclohex-1-ene; 1-methyl-4-(propan-2-ylidene)cyclohex-1-ene; 2-(isopropyl 2-methyl butanoate; 2-(1-(3,3-dimethylcyclohexyl)ethoxy)-2-methylpropyl cyclopropanecarboxylate; 2-(2-(4-methylcyclohex-3-en-1-yl)propyl)cyclopentanone; 2-(2,4-dimethylcyclohexyl)pyridine; 2-(sec-butyl)cyclohexanone; 2-(tert-butyl)cyclohexyl acetate; 2,2,2-trichloro-1-phenylethyl acetate; 2,2,5-trimethyl-5-pentylcyclopentanone; 2,2-dimethyl-2-pheylethyl propanoate; 2,4,6-trimethyl-4-phenyl-1,3-dioxane; 2,4,6-trimethylcyclohex-3-enecarbaldehyde; 2,6,10-trimethylundec-9-enal; 2,6-dimethylhept-5-enal; 2,6-dimethylheptan-2-ol; 2-cyclohexylidene-2-(o-tolyl)acetonitrile; 2-cyclohexylidene-2-phenylacetonitrile; 2-ethyl-methyl-(m-tolyl)butanamide; 2-isopropyl-5-methylcyclohexanone; 2-methyl-4-methylene-6-phenyltetrahydro-2-pyran; 2-methyldecanenitrile; 2-pentylcyclopentanone; 3-(3-isopropylphenyl)butanal; 3-(4-(tert-butyl)phenyl)-2-methylpropanal; 3-(4-ethylphenyl)-2,2-dimethylpropanal; 3-(4-isobutyl-2-methylphenyl)propanal; 3-(4-isobutylphenyl)-2-methylpropanal; 3,7-dimethyloct-6-en-1-ol; 3,7-dimethyloct-6-enal; 3,7-dimethylocta-1,6-dien-3-ol; 3,7-dimethyloctan-3-ol; 4-(4-methylpent-3-en-1-yl)cyclohex-3-enecarbaldehyde; 4-(tert-pentyl)cyclohexanone; 4,4a,5,9b-tetrahydroindeno[1,2-d][1,3]dioxine; 4-cyclohexyl-2-methylbutan-2-ol; 5-(sec-butyl)-2-(2,4-dimethylcyclohex-3-en-1-yl)-5-methyl-1,3-dioxane; 5-tert-butyl-2-methyl-5-propyl-2-furan; 6-(sec-butyl)quinoline; 6,8-dimethylnonan-2-ol; 6-ethyl-3-methyloct-6-en-1-ol; 8-(sec-butyl)-5,6,7,8-tetrahydroquinoline; 8,8-dimethyl-1,2,3,4,5,6,7,8-octahydronaphthalene-2-carbaldehyde; allyl 2-(cyclohexyloxy)acetate; dec-4-enal; dec-9-en-1-ol; dodec-2-enal; dodecanal; dodecanenitrile; ethyl 2-ethyl-6,6-dimethylcyclohex-2-enecarboxylate; ethyl 2-methylpentanoate; ethyl octanoate; hex-3-en-1-yl methyl carbonate; hexyl 2-hydroxybenzoate; methyl 3-oxo-2-pentylcyclopentaneacetate; oxacyclohexadecan-2-one; and the like.

Further disclosed is an apparatus for carrying out the disclosed method. The apparatus comprises a sealed sample vessel comprising a penetrable surface and an interior comprising a sample region and a headspace region. The sample vessel may be any suitable autosampler vessel or vial, and may be made from glass, polymer or composite material. The sealed sample vessel includes a surface that is penetrable or pierceable, such as a self-sealing membrane or septum that is capable of being penetrated for the purpose of insertion and withdrawal of contents from the interior of the sample vessel. The liquid sample region is located in the interior of the sample vessel and near the bottom of the sample vessel and is configured to receive and hold a desired volume of a liquid sample. The headspace region of the sample vessel is located within the interior of the sample vessel and above the liquid sample region.

The apparatus includes an inlet configured for passing through the penetrable surface of the sample vessel and which is in fluid communication with the interior of the sealed sample vessel and the environment external to the interior of the sample vessel. The apparatus further includes an outlet configured for passing through the penetrable surface of the sample vessel and which is also in fluid communication with the interior of the sealed sample vessel and the environment external to the interior of the sample vessel. The inlet structure comprises an elongated structure configured to receive an inert carrier gas from a source of carrier gas and to permit passage of the inert carrier gas through the inlet into the interior of the sealed sample vessel. The outlet comprises an elongated structure configured to receive inert carrier gas and gaseous analyte from the headspace region of the sealed sample vessel and to permit passage of the inert carrier gas and gaseous analyte through the outlet to a desired location external to the sealed sample vessel, such as a collection vessel. According to certain embodiments, both the elongated inlet and outlet structures may be selected from the group consisting of a cannula, conduit, cylinder, duct, hose, line, needle, passage, or tube. According to certain embodiments, the elongated inlet and outlet structures comprise a needle with a tip configured for penetrating or otherwise piercing a surface of the sealed sample vessel.

The apparatus further includes an analyte collection vessel that is in fluid communication with the interior of the sample vessel via the elongated outlet structure. The analyte collection structure is configured to accommodate an adsorbent material or structure that is coupled with the collection vessel for adsorbing gaseous flavour components of the analyte that is carried to the analyte collection vessel from the interior headspace region of the sample vessel by the inert carrier gas through the elongated outlet structure. The collection vessel may be coupled to a heating means to heat the adsorbed flavour components of the analyte to desorb such flavour components from the adsorbent material so that the continuously flowing inert carrier gas can carry the desorbed flavour components into the gas chromatograph.

A gas chromatograph is placed in fluid communication with the collection vessel. The gas chromatograph includes a sample inlet to a gas chromatography column of the chromatograph that is configured to receive a flow of inert carrier gas containing the gaseous sample comprising the flavour components of the flavour compound or composition being evaluated that have been desorbed from the adsorbent material in the collection vessel, and to permit passage of the sample into the chromatography column.

The gas chromatograph also includes means for concentrating or re-focusing the gaseous sample (ie, the headspace vapor) near the inlet of the chromatography column and/or along the proximal length or section of the chromatography column near the inlet. According to certain embodiments, a suitable cooling means is provided to concentrate the sample in a smaller volume of vapor at or near the chromatography column inlet. The cooling means may include means for delivering a flow or stream of cooling liquid or gas to the inlet or the column and/or to a section of chromatography column adjacent to and immediately downstream from the column inlet. Without limitation, the cooling means may comprise a suitable length of jacket, sheath or tubing that is positioned around a desired length of chromatography column located near the inlet of the column. The jacket, sheath or tubing may comprise any material that is capable of being positioned about the exterior surfaces of a section of gas chromatography column, receiving a cooling substance, and permitting flow of such cooling substance along the length of the tubing. One end of the jacket, sheath or tubing is connected to, and is in fluid communication with, a source of cooling substance. The cooling substance may be any cooling or chilling gas or liquid capable of concentrating the gaseous sample (ie, the headspace vapor) for gas chromatography. According to certain embodiments the cooling or chilling substance is a cryogenic fluid. Cryogenic fluids are cooling or chilling fluids that are liquids at or below a temperature of about −150° C. Suitable cryogenic fluids that may be delivered to chromatography column jacket include, for example, but are not limited to, liquid argon, liquid carbon dioxide, liquid helium, liquid nitrogen, and the like. According to certain embodiments, the cryogenic fluid is liquid nitrogen. According to other embodiments, the cryogenic fluid is liquid carbon dioxide. The cryogenic cooling or chilling of the sample with the cryogenic fluid can be carried out at any cryogenic temperatures that are suitable to sufficiently chill or cool the sample for subsequent chromatography. By way of illustration, and not in limitation, the cryogenic cooling or chilling of the sample with the cryogenic fluid can be carried out at temperatures at or below −150° C., at or below −160° C., at or below −170° C., at or below-180ºC, at or below −190° C., or at or below −200° C., or lower temperatures. The apparatus includes controls in which to turn on and off the supply of cryogenic cooling fluid delivered to the column. After the cooling step is completed and the sample has been sufficiently concentrated, the gas chromatography column is permitted to return to the desired oven temperature and the sample is re-vaporized for separation in the downstream section of the column. The gas chromatograph includes a detector downstream from the chromatography column for identifying separated flavour components of the analyte and carrying out both qualitative and quantitative analysis of the flavour components. A mass spectrometer is coupled with, and is in fluid communication with, the gas chromatograph.

Without limitation, a suitable gas chromatography system is Agilent 7890B GC system commercially available from Agilent Technologies. Without limitation, a mass spectrometry system is Agilent 5975B Inert XL EI/CI MSD system commercially available from Agilent Technologies.

The disclosed method can be used to determine loss of flavour attributable to volatile loss and chemical oxidation of any encapsulated flavour compound or flavour composition that is not 100% retained within a microcapsule.

The present disclosure provides a method for determining headspace concentration of volatile flavour components and their respective degradation products and monitoring them as they increase or decrease over time under accelerated storage conditions. The method enables the determination of the approximate shelf life of an encapsulated flavour that takes into account both volatile loss and loss due to chemical oxidation of flavour components. The methods permits the quantification of volatile flavour components and their degradation products by the same method and at the same time. The use of the disclosed method enables the determination of flavour shelf life by considering both the flavour precursor and the corresponding degradation product(s).

### EXAMPLES

The following examples are given solely for the purpose of illustration and are not to be construed as limitations of the present invention, as many variations of the invention are possible without departing from the spirit and scope of the present disclosure.

### Example 1—Quantification of Limonene

An encapsulated limonene flavour was prepared by dissolving Capsul™ modified starch (Ingredion) in deionized water. Maltodextrin (STAR-DRI 100; Tate & Lyle) and sugar were and thoroughly mixed. Limonene (Berje, Inc.) flavour was added and the entire mixture was mixed with high shear to reduce droplet size to about 1 micron. The mixture was spray dried with a dryer at an inlet temperature of 180ºC and an outlet temperature of 86° C. An encapsulated limonene flavour was evaluated for degradation and flavour loss over time using the disclosed method. 10 mg of spray dried microcapsules of encapsulated limonene flavour (25% load) was dissolved in water. The dissolved sample was transferred to a sealed autosampler vial having a penetrable membrane or septum. The dissolved sample was heated with agitation at 32° C. and 500 RPM for 2 minutes in which a gaseous analyte was formed in the headspace region of the sample vial.

The gaseous analyte was transferred from the autosampler vessel to a collection vessel or trap by flowing inert nitrogen gas from a source of gas through an elongated inlet needle that is in fluid communication with the source of nitrogen gas and headspace region of the interior of the autosampler vessel, and through an elongated outlet needle in fluid communication with the headspace region of the autosampler vessel and the interior of the collection vessel. The parameters of the analyte collection step were as follows:
Carrier Gas: Nitrogen
Flow Rate: 50 ml/minute
Temperature: 30° C.
Time: 10 minutes
Collection Volume: 500 ml

The autosampler used in this example was the Gerstel Multipurpose Sampler MPS2 equipped with a dynamic headspace sampling accessory and Gerstel sample needles.

The collected sample analyte was diluted 200:1 with inert air for separation by gas chromatography and was introduced into the inlet end of the gas chromatography column of the chromatograph. The temperature profile of the chromatographic separation was as follows:

| | Rate | Temperature | Hold Time | Run Time |
|---|---|---|---|---|
| | (° C./min) | (C. °) | (min) | (min) |
| Initial | | 40 | 2 | 2 |
| Ramp 1 | 5 | 50 | 0 | 4 |
| Ramp 2 | 1 | 70 | 0 | 24 |
| Ramp 3 | 20 | 150 | 10 | 38 |

| | |
|---|---|
| Post Run Temp | 50° C. |

As used in this specification any reference to the phrases “one embodiment” or “an embodiment” means that a particular element, feature, structure, process step, or characteristic described in connection with the embodiment is included in at least one embodiment. The particular element, feature, structure, process step, or characteristic may, in fact, be included in more than one embodiment disclosed herein. Furthermore, the use of the phrase “in one embodiment” in various places in the specification do not necessarily all refer to the same embodiment.

As used in this specification, the terms “comprises,” “comprising,” “contains,” “containing,” “includes,” “including,” “has,” or “having,” are open-ended expressions and are intended to cover methods, processes, steps, products, apparatus, or systems that comprise a recited list of components, elements, and features, and any and all additional components, elements and features that are not expressly recited.

As used in the present specification, the term “or” refers to the use of an inclusive “or” and not to an exclusive “or”. For example, the phrase “A or B” is satisfied by any one of the following: A is present (element or method step) and B is not present (element or method step), A is not present (element or method step) and B is present (element or method step), and both A and B are present (element or process step).

As used in the present specification, “a” or “an” is employed to describe components, elements, features and method/process steps of various illustrative embodiments disclosed herein. The use of “a” or “an” should be interpreted to include one or more than one.

As used in the present specification, any of the terms “preferably,” “commonly,” and “typically” are not intended to, and do not, limit the scope of disclosed methods, processes or techniques. Rather, these terms are merely intended to identify particular aspects of an embodiment or to emphasize alternative or additional features that may or may not be utilized in a particular embodiment.

In the present disclosure, the term “about” used in connection with a value is inclusive of the stated value and has the meaning dictated by the context. For example, it includes at least the degree of error associated with the measurement of the particular value. One of ordinary skill in the art would understand the term “about” is used herein to mean that an amount of “about” of a recited value produces the desired degree of effectiveness in the compositions and/or methods of the present disclosure. One of ordinary skill in the art would further understand that the metes and bounds of “about” with respect to the value of a percentage, amount or quantity of any component in an embodiment can be determined by varying the value, determining the effectiveness of the compositions or methods for each value, and determining the range of values that produce compositions or methods with the desired degree of effectiveness in accordance with the present disclosure.

It should be understood that when a range of values is described in the present disclosure, it is intended that any and every value within the range, including the end points, is to be considered as having been disclosed. For example, “a range of from 50 to 100” of a component is to be read as indicating each and every possible number along the continuum between 50 and 100. It is to be understood that the inventors appreciate and understand that any and all values within the range are to be considered to have been specified, and that the inventors have possession of the entire range and all the values within the range.

While the method of detecting flavour loss from a sample flavour has been described above in connection with certain illustrative embodiments, including those embodiments shown in the various drawing figures, it is to be understood that other embodiments may be used or modifications and additions may be made to the described embodiments for performing the same function of the present embodiments without deviating therefrom. Further, all embodiments disclosed are not necessarily in the alternative, as various embodiments of the invention may be combined to provide the desired characteristics. Variations can be made by one having ordinary skill in the art without departing from the spirit and scope of the disclosure. Therefore, the present disclosure should not be limited to any single embodiment, but rather construed in breadth and scope in accordance with the recitation of the attached claims.

1. A method for detecting flavour loss from a flavour compound comprising:
introducing a liquid sample containing a flavor compound into a sealed sample vessel;
heating the liquid sample containing the flavour compound to produce a gaseous analyte;
flowing an inert carrier gas through the sealed sample vessel;
collecting the gaseous analyte;
separating flavour components derived from the flavour compound contained in the gaseous analyte;
identifying the separated flavour components of the analyte; and
quantifying the quantity of each of the separated flavour components.

2. The method of claim 1, wherein the flavour compound is encapsulated within an encapsulant.

3. The method of claim 2, comprising at least partially dissolving the flavour compound in a fluid to provide a liquid sample containing the dissolved flavour compound prior to the heating step.

4. The method of claim 3, comprising at least partially dissolving the flavour compound in a fluid to provide a liquid sample containing the dissolved flavour compound and introducing the liquid sample containing the dissolved flavour compound into the sealed sample vessel prior to the heating step.

5. The method of claim 3, comprising introducing the flavour compound into the sealed sample vessel and at least partially dissolving the flavour compound in a fluid to provide a liquid sample containing the dissolved flavour compound within the sealed sample vessel prior to the heating step.

6. The method of claim 3, wherein the fluid comprises a liquid solvent.

7. The method of claim 6, wherein the liquid solvent comprises water.

8. The method step of claim 4, wherein the heating step comprises heating with agitation.

9. The method of claim 1, wherein the step of flowing the inert gas through the sealed sample vessel comprises flowing the inert gas into from the environment external to the sealed sample vessel into the headspace of the sealed sample vessel through an inlet.

10. The method of claim 9, wherein the inlet comprises a structure selected from the group consisting of a cannula, conduit, cylinder, duct, hose, line, needle, passage, or tube, wherein the structure penetrates the sealed sample vessel.

11. The method of claim 1, wherein the liquid sample comprises the flavour compound in an immiscible fluid.

12. The method of claim 1, wherein the flavour compound comprises a dried powder having a water-miscible outer shell and a water-immiscible inner core.

13. The method of claim 12, wherein the outer water-immiscible core comprises an oil containing the flavour compound.

14. The method of claim 7, wherein the step of at least partially dissolving the flavour compound in a fluid comprises dissolving the outer water-miscible shell leaving behind oil droplets containing the flavor compound dispersed within the water.

15. The method of claim 2, further comprising mechanically breaking apart, comminuting, cracking, crushing, cutting, dividing, fracturing, grating, grinding, or splitting the encapsulated flavour compound to release the flavour components.

16. The method of claim 1, wherein flowing an inert carrier gas through the sealed sample vessel comprises continuously flowing the inert carrier gas into the headspace region of the sealed sample vessel and the flow of the inert carrier gas continuously collects flavour components within the headspace region for a desired period of time.

17. The method of claim 1, wherein the step of collecting the gaseous analyte comprises flowing the inert carrier gas containing gaseous analyte from the headspace region of the sealed sample vessel to a collection vessel through the outlet that is in fluid communication with the collection vessel.

18. The method of claim 17, wherein the collection vessel includes an adsorbent for adsorbing the flavour components of the gaseous analyte.

19. The method of claim 18, wherein the headspace components are collected on the adsorbent and subsequently desorbed for transfer into a gas chromatography column.

20. The method of claim 18, wherein the adsorbent material is selected from carbon molecular sieves, spherical graphitized polymer carbon, graphitized carbon black, and porous polymer resins.
